(19) United States
(12) Patent Application Publication
(10) Pub. No.: US 2011/0034998 A1
(43) Pub. Date: Feb. 10, 2011

(54) ANNULOPLASTY TUBES

(76) Inventor: Kassem Ashe, Mississauga (CA)

Correspondence Address: NASSER ASHGRIZ, 7805 Bayview Ave., # 1510, Thornhill, ON, L3T 7N1, CA

(21) Appl. No.: 12/537,754

(22) Filed: Aug. 7, 2009

(51) Int. Cl. A61F 2/24

(52) U.S. Cl. 623/2.36

(57) ABSTRACT

A mitral valve annuloplasty tube and method for implanting the same to treat mitral valve regurgitation by establishing new mitral valve annulus. The method comprising of cutting and detaching the leaflets of a mitral valve and displacing them to a different location, preferably to an upstream location of the blood flow, in order to compensate for the height of the valve prolapse. The displacement zone being filled with the present annuloplasty tube.

### FIELD OF THE INVENTION

The present invention relates to a method and apparatus for establishing a new mitral valve annulus in the place of native mitral valve using an annuloplasty tube.

### BACKGROUND OF THE INVENTION

The present invention relates to the repairment of the mitral valve using a novel annuloplasty tube and a procedure for its implantation to treat mitral valve regurgitation, a cardiac abnormality where the mitral valve of the heart does not close properly.

Heart is divided into the right and the left sides, which are separated by a septum. Heart has four chambers as illustrated in FIG. 1: right atrium 1, right ventricle 2, left atrium 3 and left ventricle 4. The blood flow in the heart is regulated by four valves, namely, pulmonary valve 5, aortic valve 6, tricuspid valve 7, and mitral valve 8. The right side of the heart pumps blood from the heart to the lungs through the pulmonary artery. The left side of the heart pumps blood to other parts of the body through aorta. The pumping cycle begins when low oxygenated blood returns to heart through superior and inferior vena cavae to fill the right atrium. When right atrium is filled with blood, it contracts opening the tricuspid valve and blood is pumped into the right ventricle of the heart. When right ventricle is full of blood, the tricuspid valve closes, preventing back flow of blood into the right atrium. Then the blood is pumped into the pulmonary artery and to the lungs. The pulmonary valve is then closed to prevent backflow of blood to the right ventricle. Oxygen rich blood returns from the lungs through the pulmonary veins, and fills the left atrium. The left atrium contracts, opening the mitral valve, and the blood is pumped to the left ventricle. Meanwhile, a new contraction is taking place in the right atrium of the heart. When the left ventricle is full of blood, the mitral valve closes, and prevents blood from flowing back into the left atrium.

As illustrated in FIGS. 2a and 2b the mitral valve comprises of two valve leaflets, named anterior leaflet 62 and posterior leaflet 63. The opening of the valve is surrounded by a fibrous ring known as the mitral valve annulus 64, which forms a ring around the valve leaflets. The part of the annulus connected to the anterior leaflet is called the mitral valve anterior annulus 65 and the part connected to the leaflet is called posterior annulus 66. These leaflets are prevented from prolapsing into the left atrium by the action of tendons attached to the posterior surface of the valve, called chordae tendineae 14. The inelastic chordae tendineae are attached at one end to the papillary muscles 15 and the other to the valve cusps. In a healthy heart, the mitral valve leaflets overlap during the contraction of the left ventricle and prevent back flow of the blood into the left atrium. However, any dysfunction of any of the above mentioned sections of the mitral valve can cause mitral regurgitation or the abnormal leaking of the blood through the mitral valve from the left ventricle into the left atrium of the heart. One type of prolapse is illustrated in FIG. 1, in which mitral valve 8 is not fully closed.

Primary mitral regurgitation is due to any of the diseases that affect the mitral valve. One of the more common causes of the primary mitral regurgitation is myxomatous degeneration of the valve. This is due to a genetic abnormality that results in a defect in the collagen that makes up the mitral valve. This causes a stretching out of the leaflets of the valve and the chordae tendineae. The elongation of the valve leaflets and the chordae tendineae prevents the valve leaflets from fully coapting when the valve is closed. This causes the valve leaflets to prolapse into the left atrium, thereby causing mitral regurgitation. Another common disease is Ischemic heart disease, which causes mitral regurgitation by the combination of ischemic dysfunction of the papillary muscles, and the dilatation of the left ventricle that is present in ischemic heart disease, with the subsequent displacement of the papillary muscles and the dilatation of the mitral valve annulus. Secondary mitral regurgitation is due to the dilatation of the left ventricle, causing stretching of the mitral valve annulus and displacement of the papillary muscles. This dilatation of the left ventricle can be due to any cause of dilated cardiomyopathy, including aortic insufficiency, and nonischemic dilated cardiomyopathy.

The posterior leaflet 63 is the widest around the annulus and is divided into three scallops, referred to as P1, P2, P3, as illustrated in FIG. 2b. The anterior leaflet 62 is divided into three scallops, namely, A1, A2, and A3. The distinct areas where the anterior and posterior leaflets come together at their insertion into the annulus are referred to as commissure medial 67 and commissure lateral 68. These sections must be respected when dealing with prolapse of a commissure and the corresponding anterior and posterior leaflet segment (P3 and A3) by, for example, resection, otherwise a residual regurgitation will occur from this region.

The abnormality of the mitral valve can occur in any of the six scallops or at the two commissures. As noted earlier, diseases or certain natural defects of a heart valve, may impair the full closure of the valve, resulting in regurgitation. For example, certain diseases may cause the dilation of the heart valve annulus, resulting in the distortion of the heart valve geometry or the shape of the valve. This results in an ineffective closure of the mitral valve during ventricular contraction, leading to regurgitation or leakage.

A damaged mitral valve may be replaced or repaired. Some of the common methods of repairing a damaged mitral valve include insertion of a cloth-covered ring around the valve to bring the leaflets into contact with each other (annuloplasty), removal of redundant/loose segments of the leaflets (quadrangular resection), and re-suspension of the leaflets with artificial (Gore-Tex) cords. More recently the Alfieri stitch (or “bow-tie”) has been adapted to allow open or percutaneous repair in select patients.

The procedures for repairing dilated or elongated mitral valve annulus are generally known as mitral valve annuloplasty. In this procedure, a prostheses ring (annuloplasty ring) is sutured around the base of the valve leaflets to restrict the dilated valve leaflets to restrict the dilated valve annulus. This annuloplasty ring reduces the size of the annulus allowing for more coaption to occur between the posterior and anterior leaflets. The annuloplasty rings are designed to provide sufficient rigidity to adequately support the valve annulus, or provide flexibility to resemble the natural movement of the annulus during opening and closing of the valve.

There are four types of mitral annuloplasty rings available for mitral valve annulplasty; these include a rigid ring, a flexible ring, semi flexible ring and a ring with a rigid section or one side and flexible section on the other designed to mimic the physiological motion of the native mitral annulus. These rings are made of different alloys covered with bio compatible clothing around them. There are a number of disadvantages in using these rings in annuloplasty. One is that if a rigid annuloplasty ring is used in the surgery, the shape of the mitral valve becomes rigid and loses its natural and needed inflexibility.

Another possible complication after a mitral valve repairment is a condition called Systolic anterior motion (SAM). As noted earlier, mitral valve has two leaflets, anterior and posterior, where the anterior leaflet covers about 35% of the valve circumference while the posterior leaflet covers the rest of the valve circumference. During a systole, the posterior mitral annulus shortens. This shortening, results in a phasic narrowing of mitral valve opening. In certain cases, the anterior valve leaflet may not be fully in the closed position during a systole and a portion of it may obstruct left ventricular outflow tract (LVOT). This results in a heart condition called Systolic anterior motion (SAM). This condition is a possible complication after mitral valve repair surgery. SAM results when the anterior leaflet of the mitral valve partially obstructs the left ventricular outflow tract (LVOT). Because of the rigidity in movement of the mitral valve there is a possibility of developing a new heart condition called systole anterior motion (SAM); a condition that makes the posterior leaflet to push the anterior leaflet opposite the direction of its normal/natural movement. And also the shape of the annulus is solely governed by the inflexible rigid ring.

These drawbacks of inflexible rigid rings were prevailed by the introduction of flexible, semi flexible and rigid/flexible rings. In the case of flexible rings, these rings allow the mitral valve annulus to move and flex in its natural way with the movement of the heart. A semi flexible or combination of rigid/flexible rings cannot retain their flexibility after few months or years after the annuloplasty. This loss of flexibility disrupts the normal flexing movement of the left ventricle resulting in complications of the heart.

Mitral Valve Prolapse (MVP), which may occur during click murmur syndrome or Barlow's syndrome, is a valvular heart disease that effects five to ten percent of world population. Mitral valve leaflets are shaped as parachutes and are attached to the inner valve of the left ventricle by series of strings called “chordae” 14. As discussed earlier these leaflets prevent the back flow of blood from left ventricle into the left atrium. In case of mitral valve prolapse, when the ventricles contract, the leaflets prolapse allows the leakage of blood through the valve opening. Mitral valve prolapse occurs when the mitral valve leaflets are significantly displaced above the mitral annulus points (in many cases more than 2 mm displacement results in prolapse).

When any one of the leaflets with sections A1, A2, A3 or P1, P2, P3 or commissure medial or commissure lateral are bellowed or prolapsed, coaption between the two segments does not occur, resulting in the leakage of blood into left ventricle. There are procedures in prior art which can be used to correct this abnormality. For example, if segment P2 does not co-apt with anterior A2, then the segment P2 can be cut out and the other two ends P1 and P3 are joined together. This new segment looks like a single segment and it can coapt with the anterior segments of A1 A2 and A3 to prevent leakage. Another procedure is to stitch a new chordae (i.e. Gortex) to the valve and bring back the prolapsed segment to its original point for co-aption to occur.

In extreme cases, all the segments may prolapse. Two main surgical methods are currently being practiced in order to repair these abnormalities. The first surgical method is shortening of papillary muscles. These muscles are cut from the bottom, in order to reduce their height This results in pulling/tightening of the chordae, which brings back the prolapsed leaflets to their normal state. Although, this operation is possible, it is very difficult and the results are not reproducible. The papillary muscles are jelly type in texture and are very difficult to suture. The other surgical method is by adding new chordate (i.e. Gortex) and by pulling the prolapsed A1, A2 and A3, and P1, P2 and P3, and stitching them onto the papillary muscles to bring the leaflets to their normal position. The major difficulty with this technique is that it needs more than 20 to 30 new chordae to bring back the leaflets to the plane of the mitral valve annulus. This is a very complicated technique and needs a high level of expertise for successful surgery.

A review of the prior art clearly points to the need for a new device and a method for the repairment of mitral valve that has a lower risk of complications, is easier to perform the operation and to produce results in the case where all the segments of anterior and posterior leaflets are prolapsed.

### SUMMARY OF THE PRESENT INVENTION

A new device for the repairment of the Mitral valve and a method of implanting it is disclosed. The new device comprises of a presized tube having substantially the same shape as the native annulus, and having an upper and a lower opening. The annuloplasty tube may be pre-sized before the surgery. The height (i.e., length) of the tube depends on the height of the prolapse. The height of the prolapse can be measured before the operation by Echo, an ultrasound instrument to assess the heart functions. This annuloplasty tube is used to displace and extend the location of the mitral valve annulus. The mitral valve is completely cutout of its location around the annulus and is attached to the upper opening of the tube. The lower opening of the tube is then attached to the original location of the valve. Therefore, the valve is displaced and lifted so much so that the prolapsed does not occur.

An annuloplasty ring may be used at the upper opening of the tube to construct a new annulus. An annuloplasty ring may or may not be used at the lower opening of the tube to suture the tube to the native annulus.

The method of implanting the annuloplasty tube is first to completely disconnect the anterior and posterior mitral leaflets circumferentially around the annulus. The height of the prolapse is predetermined pre operatively or intra operatively by Echo measurements. Then, the surgeon places the tube on the native annulus and the leaflets are sutured around the annuloplasty ring on the annuloplasty tube at the inlet part of the tube, which becomes the new functional annulus. Therefore, the leaflets co-apt at their new height achieved by the annuloplasty tube. The lower end of the annuloplasty tube is sutured around the old annulus level. This surgery may allow the mitral regurgitation to stop completely.

Therefore, one object of the present invention is to provide an annuloplasty tube that establishes a new mitral valve annulus at a displaced location.

It is further object of the present invention to provide an annuloplasty tube that induces effective coaption of the anterior and posterior leaflets on the newly formed mitral valve annulus.

It is further object of the present invention to provide an alternative method to the shortening of the papillary muscles to eliminate prolapse.

It is further object of the present invention to provide annuloplasty tube and method of implanting the annuloplasty tube that does not alter the movement of left ventricle when it contracts or relaxes.

It is further object of the present invention to provide a method that can replace the method of suturing the leaflets onto the papillary muscles.

It is further object of present invention to provide an annuloplasty tube and the surgical method which can prevent occurrence of systole anterior motion(SAM) after surgery.

It is further object of the present invention to provide an annuloplasty tube that is sufficiently flexible to allow for the newly established mitral valve annulus to flex in different planes without destabilizing the regular heart function.

It is further object of the present invention to provide an annuloplasty tube comprising of annuloplasty rings that can be flexed like the natural mitral valve annulus. Said rings may be rigid, flexible, semi flexible or combination of rigid and flexible rings.

It is further object of the present invention to have an annuloplasty tube comprising of an annuloplasty ring connected to its outlet opening, whereas the ring can be readily adjusted to the circumference of the native annulus; the annuloplasty tube and the annuloplasty rings are bio-compatible with human body.

It is further object of the present invention that the height and the diameter of the annuloplasty tube can be adjusted prior to the surgery or during the surgery.

It is further object of the present invention to have a height adjustable means built in the annuloplasty tube, so that the height of the tube can be adjusted immediately during the operation.

### BRIEF DESCRIPTION OF THE DRAWINGS

In the figures, which illustrate, by way of example only, embodiments of the present invention,

FIG. 1 represents the surgical anatomy of a heart, and a prolapsed mitral valve;

FIGS. 2a and 2b represents the surgical anatomy of a mitral valve;

FIG. 3 represents the cutaway aerial view of a heart;

FIGS. 4a and 4b represent lateral views of a preferred embodiment of the mitral valve annuloplasty tube of present invention with annuloplasty rings, disassembled and assembled, respectively;

FIG. 5 represents lateral views of a preferred embodiment of the mitral valve annuloplasty tube of present invention having tethers;

FIG. 6 represents lateral views of a preferred embodiment of the mitral valve annuloplasty tube having different top and bottom sizes;

FIG. 7 represents prolapsed leaflets and a defined prolapsed height H;

FIG. 8 represents disconnecting the anterior and posterior leaflets;

FIGS. 9a, 9b, and 9c represents the lateral cross sectional view of the circumferentially disconnected leaflets, lifted leaflets, and installed annuloplasty tube; and

FIG. 10 represents the formation of new functional annulus with attached leaflets.

### DETAILED DESCRIPTION

The following description of the preferred embodiments of the invention are exemplary, rather than limiting, and many variations and adaptations are within the scope of the invention.

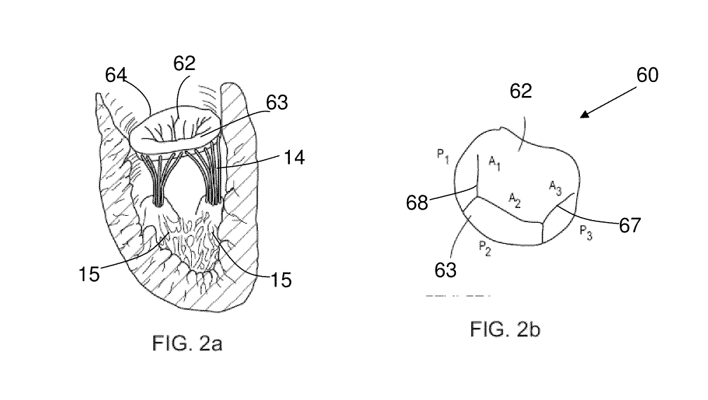

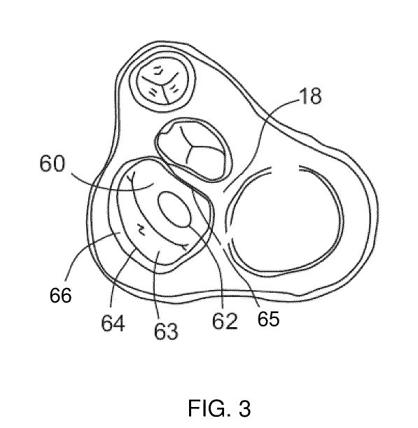

The present invention is described in greater details with reference to FIGS. 2 and 3. The mitral valve 60 comprises of two leaflets, a larger anterior leaflet 62 and smaller posterior leaflet 63, these leaflets acts like doors, opening and closing by the pressure induced by the heart. The mitral valve annulus 64 holds both the anterior leaflet 62 and posterior leaflet 63. The mitral valve annulus 64 is circumflexed around with annulus posterior 66 and anterior 65. The present device is used to repair a Mitral valve having a prolapse. This is achieved by implanting an annuloplasty tube inside the heart.

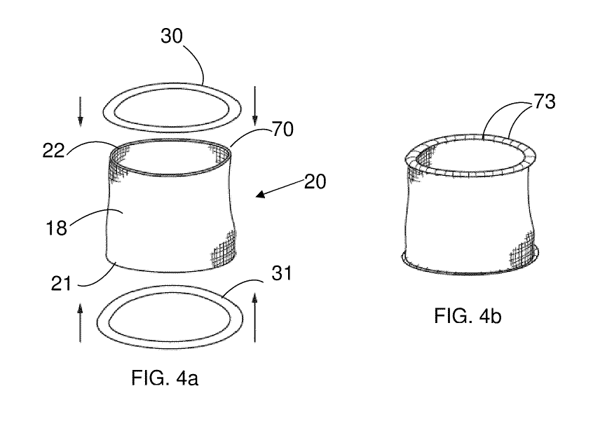

One embodiment of annuloplasty tube 20 of the present invention is illustrated in FIG. 4a. The main body of the tube 18 is substantially smooth, continuous, and generally curvilinear with no abrupt transitions. The tube is made of a biocompatible material, such as Dacron cloth or Cortex. The annuloplasty tube 20 has a bottom (outlet) end 21, which has substantially the same shape as the native mitral valve annulus 64, and a top (inlet) end 22, which defines a new annulus where the anterior leaflet 63 and posterior leaflet 62 attach to form the new mitral valve annulus. The tube can be flexible, semi-flexible, or rigid. Since the tube length is relatively short, few millimeters (5-20 mm), it will not collapse. However, in order to provide more rigidity to the tube, the top end of the tube may be made rigid. This can be achieved by attached an annuloplasty tube to the top end.

The tube may have one or two annuloplasty rings 30 and 31 connected to the upper and lower ends, respectively. A tube having annuloplasty rings is illustrated in FIG. 4b. The annulpplaty rings can be chosen from any commercially available annuloplasty rings suitable for the specific patient. Most annuloplasty rings have an oval or somewhat D-shaped configuration with a relatively straight anterior section opposite a curved posterior section. A plurality of knotted suture loops 73 (FIG. 4b) are typically used to secure the ring to the mitral annulus, although other fasteners such as staples, or the like may be used. The rings preferably include an inner ring body and an outer sewing sheath that permits the ring body to be sutured into the mitral annulus. The sewing sheath should be sufficiently porous and/or flexible to permit sutures to be passed therethrough. Detailed description of the annuloplasty rings are not provided here and are incorporated by reference.

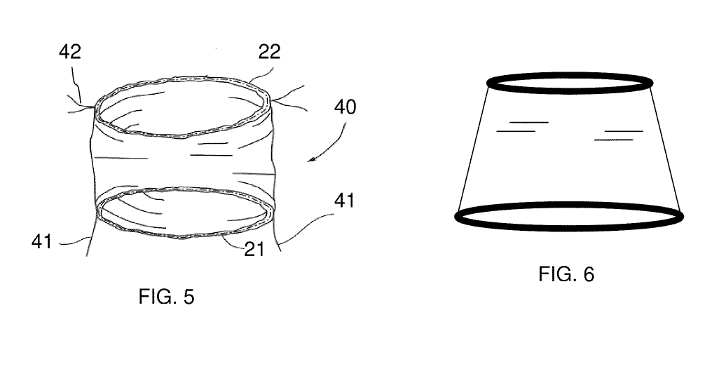

Another embodiment of the annuloplasty tube 40 having a height adjusting means 41 and/or a diameter adjusting means 42 is illustrated in FIG. 5. Once the height of the prolapse is known, the height of the annuloplasty tube can be adjusted before or after the implantation, which saves time and complexities during the surgery. This is achieved by having inbuilt tethers 41. By pulling the tethers longitudinally, the height of the hollow tube 40 can be adjusted. The tube has tethers 41 and/or 42 protruding from the ends, which can shorten the tube by pulling on the tethers and/or reduce the top and bottom diameters of the tube, respectively. Preferably, only a small section of the tube (about 20%) has adjustable tethers. Therefore, small changes during the operation can be achieved. Larger changes may require cutting of the tube.

The top and bottom diameters of the tube may be of different sizes as illustrated in FIG. 6. This may be helpful in some cases in which the leaflets may hit walls of the tube during the opening cycle. The exact dimensions of the tube have to be determined based on the size of the annulus and the length of the leaflets.

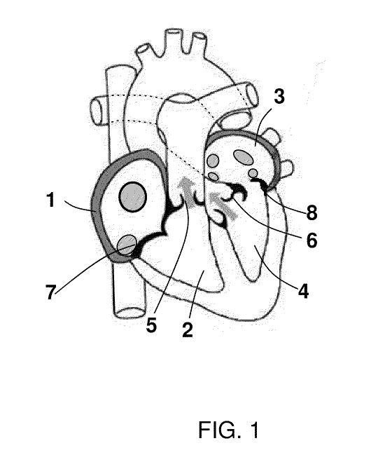

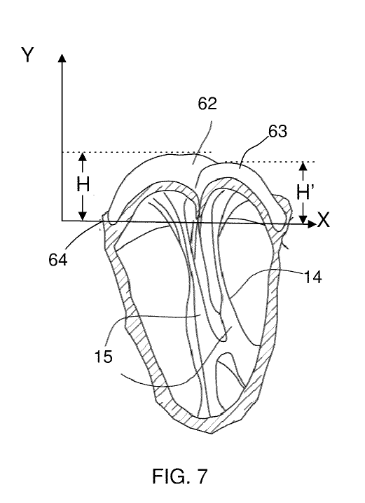

FIG. 7 illustrates the pan segmental prolaps of mitral valve, wherein leaflets are bellowing above the mitral valve annulus 64. In the case of mitral valve prolapse, when the ventricles contract, the prolapse allows the leakage of blood through the valve opening as illustrated in FIG. 1. Mitral valve prolapse occurs when the mitral valve leaflets are displaced too much (typically more than 2 mm) above the mitral annulus points. The prolapsed height H is defined as the height of the bellowing of the leaflets above the plane X, which is defined as the normal plane of co-aptation of leaflets for competence.

Replacement of native mitral valve annulus 64 with annuloplasty tube begins by measuring the height of prolapse H of the anterior leaflet 62 and H′ of posterior leaflet 63 by using echocardiogram measurements. The height of the annuloplasty tube 20 is adjusted to match the heights H and H′ of prolapsed leaflets.

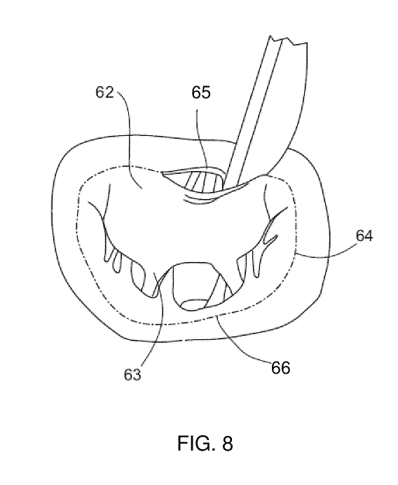

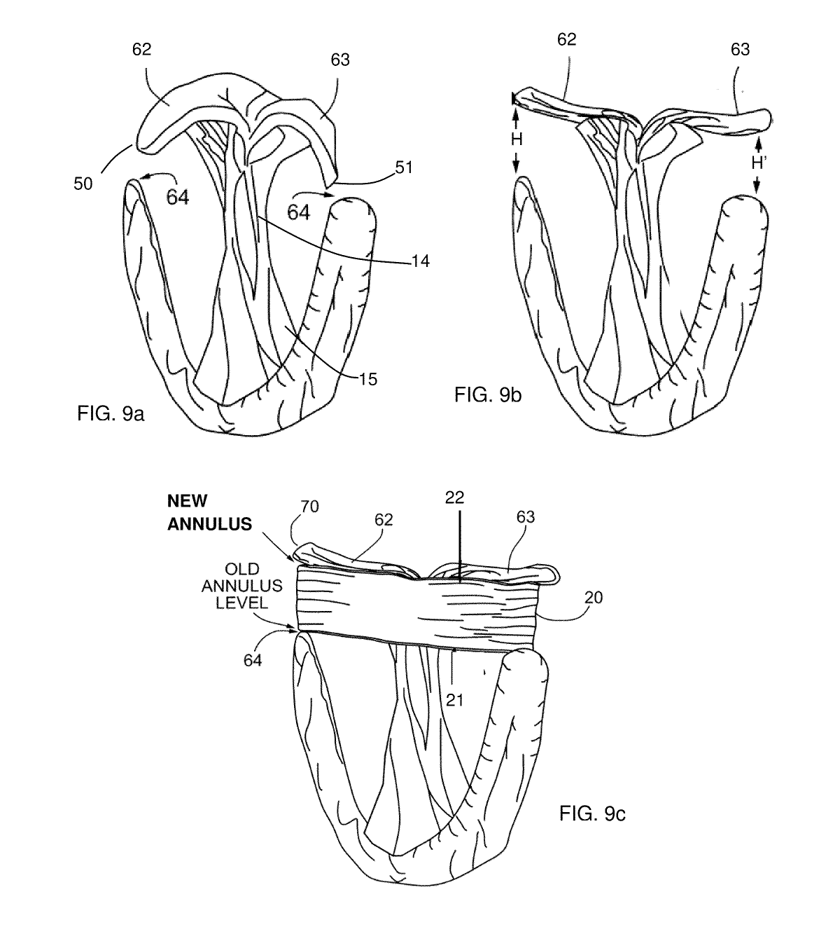

As illustrated in the FIG. 8 the native mitral valve 64 is dissected from the heart. Anterior leaflet 62 and posterior leaflet 63 are circumferentially disconnected all the way around from the annulus 64 across the posterior points 66 and anterior points 65, resulting in free edges 50 from anterior leaflet 62 and 51 from posterior leaflet 63. As illustrated in FIG. 9a, the anterior leaflet 62 and posterior leaflet 63 remain firm and connected to the native chordae tendineae 14 further connecting to the papillary muscles 15. As illustrated in FIG. 9b The detached leaflets are then raised to a height H and H′ such that during operation there is co-apt between the leaflets with no prolapse. A annuloplasty tube is then implanted to build the opening between the native annulus and the leaflets, as illustrated in FIG. 9c.

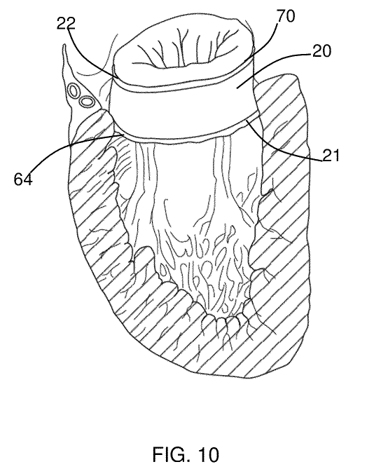

FIG. 10 illustrates the device 20 passed through the leaflets. The bottom end 21 of the tube 20 is sutured onto the native annulus 64. The bottom end 21 may be directly sutured to the native annulus or it may be first connected to an annuloplasty ring and then the ring be sutured to the annulus. The free ends of the anterior leaflet 62 and the posterior leaflet 63 are sutured to the top end of the tube 22. Again the leaflets may be directly connected to the tube or they may be first connected to an annuloplasty ring, which is sutured to the top end of the ring.

The tube having anterior leaflet 62 and posterior leaflet 63 on its top end 22 becomes the new functional annulus 70 without any prolapse. By performing this surgery, the two leaflets, anterior leaflet 62 and posterior leaflet 63, co-apt at a height H above the old natural annulus, to prevent regurgitation. The device 20 and method of implanting the device disclosed here in this invention will maintain the continuity of the novel mitral valve annulus and the papillary muscles.

It will be appreciated by those of skilled in the relevant art that various modifications or changes may be made to the examples and embodiments of the invention described in this provisional application, without departing from the intended spirit and scope of the invention. In this regard, the particular embodiments of the invention described herein are to be understood as examples of the broader inventive concept disclosed in this application.

What is claimed is:

1. An annuloplasty tube for repairing mitral valve regurgitation in patients with heart valve prolapse, said annuloplasty tube comprising:
a bio compatible tube having a top end, a bottom end and a height;
the bottom end is shaped and sized to attach to the annulus of a mitral valve;
the top end is shaped and sized to receive the detached leaflets of a mitral valve;
the height of said tube is substantially the same as the predetermined height of a prolapse,
whereby the leaflets of the mitral valve are cutout circumferentially and are separated from the native annulus, said cutout valve is then secured to the top end of the annuloplasty tube, whereby the valve is displaced to a different height than that of native annulus.

2. The annuloplasty tube of claim 1, having a an anterior section adapted to match and to connect to the anterior side of the mitral valve annulus, and a posterior section adapted to match and to connect to the posterior side of the mitral valve annulus.

3. The annuloplasty tube of claim 1, wherein the top end of the tube comprises of a rigid material.

4. The annuloplasty tube of claim 1, wherein the top end of the tube comprises of a rigid material and the bottom end comprises of a flexible material and the rigidity of the tube body gradually decreases from the top end to the bottom end.

5. The annuloplasty tube of claim 1, wherein the top end of the tube further comprises of an annuloplasty ring.

6. The annuloplasty tube of claim 1, wherein the bottom end of the tube comprises of a flexible material.

7. The annuloplasty tube of claim 1, wherein the bottom end of the tube further comprises of an annuloplasty ring, said ring having securing means for securing the ring on the inner surface of the annulus.

8. The annuloplasty tube of claims of 1, further comprising a securing means selected from the group consisting of sutures, staples, and mechanical fasteners.

9. The annuloplasty tube of claims 5, wherein said annuloplasty rings are selected from a group consisting of rigid, flexible, semi flexible, or any combination of rigid/flexible rings.

10. The annuloplasty tube of claim 1, wherein the height of the annuloplasty tube is between 5 mm to 20 mm.

11. The annuloplasty tube of claim 1, wherein said tube is made of polyethylene terephthalate cloth or other bio compatible and suturable material.

12. The annuloplasty tube of claim 1, wherein a section of said tube having means to adjust its height, whereby the height adjustment can be done quickly during the operation.

13. The annuloplasty tube of claim 12, wherein said adjusting means are inbuilt tethers made of biocompatible string like material.

14. The annuloplasty tube of claim 13, wherein said tethers pass longitudinally across the tube.

15. The annuloplasty tube of claim 1, wherein said tube having means to adjust the diameter of the top end and the diameter of the bottom end, whereby the opening diameters of the tube can be adjusted to properly fit inside the native annulus and to receive the leaflets.

16. The annuloplasty tube of claim 1, wherein the diameter of the top end is smaller than the diameter of the bottom end.

17. The annuloplasty tube of claim 1, wherein said tube having varying heights across its diameter to match the varying heights of prolapse in the Mitral valve of a patient.

18. A method of repairing mitral valve regurgitation in patients with heart valve prolapse, said method comprising: cutting and detaching the anterior and posterior mitral leaflets circumferentially all the way around from a native annulus;
determining the height of the prolapse;
displacing the detached leaflets to a height above the native annulus in order to match the height of the prolapse and to cause the leaflets to co-apt at the same plane, said height defining a new height;
filling the space between the native annulus and the new height with an annuloplasty tube, said tube having a top end and a bottom end;
securing the bottom end of the tube to the native annulus;
connecting an annuloplasty ring to the top end of the tube; and
securing said leaflets to the ring at the top end,
whereby the tube height corrects the prolapse so that the two leaflets touch each other.

19. A method of claim 18, further comprising adjusting the height the tube while the flow through the valve is observed to determine whether the regurgitation is inhibited.

20. A method of claim 18, further comprising adjusting the shape of the top ring to stabilize and leaflet function and inhibit prolapse.
